(19) United States
(12) Patent Application Publication
(10) Pub. No.: US 2006/0041311 A1
(43) Pub. Date: Feb. 23, 2006

(54) Devices and methods for treating facet joints

(76) Inventor: Thomas J. McLeer, Redmond, WA (US)

Correspondence Address: WILSON SONSINI GOODRICH & ROSATI, 650 PAGE MILL ROAD, PALO ALTO, CA, 94304-1050, US

(21) Appl. No.: 11/207,991

(22) Filed: Aug. 18, 2005

Related U.S. Application Data
(60) Provisional application No. 60/602,829, filed on Aug. 18, 2004.
(60) Provisional application No. 60/602,964, filed on Aug. 18, 2004.

(51) Int. Cl. A61F 2/44

(52) U.S. Cl. 623/17.11; 623/18.11; 623/908

(57) ABSTRACT

The invention discloses methods and devices for repairing, replacing and/or augmenting natural facet joint surfaces and/or facet capsules.

### CROSS-REFERENCE

This application claims the benefit of U.S. Provisional Application No. 60/602,829, to Thomas J. McLeer, filed Aug. 18, 2004, and entitled “Inlay Articulation for Facet Replacement,” and U.S. Provisional Application No. 60/602,964, to Thomas J. McLeer, filed Aug. 18, 2004, entitled “Facet or Joint Capsule Device,” the disclosures of which are incorporated herein by reference in their entireties.

### FIELD OF THE INVENTION

The present invention generally relates to devices and surgical methods for the treatment of various types of pathologies of facet joints of the spine and similar joints. More specifically, the present invention is directed to several different types of devices and methods for treating injured or diseased facet joints of the spine.

### BACKGROUND OF THE INVENTION

Back pain, particularly in the “small of the back” or lumbosacral (L4-S1) region, shown in FIG. 1, is a common ailment. In many cases, the pain severely limits a person's functional ability and quality of life. Such pain can result from a variety of spinal pathologies. Through disease or injury, the laminae, spinous process, articular processes, or facets of one or more vertebral bodies can become damaged, such that the vertebrae no longer articulate or properly align with each other. This can result in an undesired anatomy, loss of mobility, and pain or discomfort.

In many cases, the vertebral facet joints can be damaged by either traumatic injury or by various disease processes. The facet joint has been implicated as a potential cause of neck pain for persons having whiplash.

These disease processes include osteoarthritis, ankylosing spondylolysis, and degenerative spondylolisthesis. Aside from pain coming from the facets themselves, such damage to the facet joints can often result in eventual degeneration, abrasion, or wearing down of the facet joints, eventually resulting in pressure on nerves, also called “pinched” nerves, or nerve compression or impingement. The result is further pain, misaligned anatomy, and a corresponding loss of mobility. Pressure on nerves can also occur without an anatomic or functional manifestation of a disease, or pathology, at the facet joint, e.g., as a result of a herniated disc.

One type of conventional treatment of facet joint pathology is spinal stabilization, also known as intervertebral stabilization. Intervertebral stabilization desirably prevents relative motion between vertebrae of the spine. By preventing movement, pain can be reduced. Stabilization can be accomplished by various methods. One method of stabilization is spinal fusion. Another method of stabilization is fixation of any number of vertebrae to stabilize and prevent movement of the vertebrae. In addition, where compression or subsidence of the disc and/or facet joints has occurred, the physician can utilize fusion devices such as pedicle screw and rods systems, or interbody fusion cages, to elevate or “jack up” the compressed level, desirably obtaining a more normal anatomical spacing between the vertebral bodies. Various devices are known for fixing the spine and/or sacral bone adjacent the vertebra, as well as attaching devices used for fixation, are known in the art, including: U.S. Pat. Nos. 6,290,703, 5,782,833, 5,738,585, 6,547,790, 6,638,321, 6,520,963, 6,074,391, 5,569,247, 5,891,145, 6,090,111, 6,451,021, 5,683,392, 5,863,293, 5,964,760, 6,010,503, 6,019,759, 6,540,749, 6,077,262, 6,248,105, 6,524,315, 5,797,911, 5,879,350, 5,885,285, 5,643,263, 6,565,565, 5,725,527, 6,471,705, 6,554,843, 5,575,792, 5,688,274, 5,690,630 6,022,350 4,805,602 5,474,555 4,611,581, 5,129,900, 5,741,255, 6,132,430; and U.S. Patent Publication No. 2002/0120272.

Another type of conventional spinal treatment is decompressive laminectomy. Where spinal stenosis (or other spinal pathology) results in a narrowing of the spinal canal and/or the intervertebral foramen (through which the spinal nerves exit the spine), and neural impingement, compression and/or pain results, the tissue(s) (hard and/or soft tissues) causing the narrowing may need to be resected and/or removed. A procedure which involves excision of part or all of the laminae and other tissues to relieve compression of nerves is called a decompressive laminectomy. See, for example, U.S. Pat. Nos. 5,019,081, 5,000,165, and 4,210,317. Depending upon the extent of the decompression, the removal of support structures such as the facet joints and/or connective tissues (either because these tissues are connected to removed structures or are resected to access the surgical site) may result in instability of the spine, necessitating some form of supplemental support such as spinal fusion, discussed above.

### SUMMARY OF THE INVENTION

While spinal fusion has become the “gold standard” for treating many spinal pathologies, including pathologies such as neurological involvement, intractable pain, instability of the spine and/or disc degeneration, it would be desirable to reduce and/or obviate the need for spinal fusion procedures, as well as reduce the need for other procedures designed to stabilize, or preserve motion, of the spinal motion segment (including, but not limited to, facet joint repair or replacement, intervertebral disk replacement or nucleus replacement, implantation of interspinous spacers and/or dynamic stabilization devices, and/or facet injections). Desirably, a physician could treat the degenerating and/or diseased tissues prior to the point where the spinal motion segment degradation mandates treatment with a spacer, fusion implant, dynamic stabilizer and/or implantation of a replacement facet and/or intervertebral disc. In such a case, the treatment would potentially slow, halt or reverse progression of the degradation and/or disease.

The present invention includes the recognition that many spinal pathologies eventually requiring surgical intervention can be traced back, in their earlier stage(s), to some manner of a degeneration, disease and/or failure of the facet joints. Moreover, spinal fusion procedures can eventually require further surgical intervention. For example, degeneration of facet joints can result in an unnatural loading of an intervertebral disc, eventually resulting in damage to the disc, including annular bulges and/or tears. Similarly, degeneration and/or failure of a facet joint can potentially lead to slipping of the vertebral bodies relative to one another, potentially resulting in spondylolisthesis and/or compression of nerve fibers. In addition, degeneration of the facet joints themselves can become extremely painful, leading to additional interventional procedures such as facet injections, nerve blocks, facet removal, facet replacement, and/or spinal fusion. Thus, if the degenerating facet joint can be treated at an early stage, the need for additional, more intrusive procedures, may be obviated.

In a similar manner, the present invention includes the recognition that many spinal pathologies mandating repair and/or replacement of an intervertebral disc (including many of those that may be currently treated through spinal fusion, interspinous distraction and/or dynamic stabilization), can often be traced back to degeneration, disease and/or failure of the facet joints. Alteration of the facet joint biomechanics resulting from an anatomic or functional manifestation of a disease can adversely affect the loading and biomechanics of the intervertebral disc, eventually resulting in degeneration, damage and/or failure of the intervertebral disc. Accordingly, early intervention and repair, augmentation and/or replacement of the facet joints may obviate the loading conditions that eventually result in such damage to the disc.

The various embodiments disclosed and discussed herein may be utilized to restore and/or maintain varying levels of the quality or state of motion or mobility and/or motion preservation in the treated facet joint(s). Depending upon the extent of facet joint degradation, and the chosen treatment regime(s), it may be possible to completely restore the quality or state of motion across one or more of the facet joints, or restore limited motion across the facet joint(s) to reduce or obviate the need for further treatment of the spinal motion segment.

An embodiment of the invention includes a facetjoint restoration device for use in a restoring a target facet joint surface comprising: a first surface configured to articulate with respect to an opposing surface comprising one of a facet joint surface or a facet joint restoration device surface; and a second surface configured to engage a surface of the target facet joint. The second surface can be configured in various embodiments in a variety of ways. For example the second surface can be configured to promote bony in-growth, adapted to secure the restoration device to the surface of the target facet joint, or adapted to provide an anchoring mechanism. Various materials are suitable for manufacturing the facet joint restoration device including, naturally occurring materials adapted to form a device, ceramic, metal, or polymer, or combinations thereof. In an embodiment of the invention, the devices are designed to restore the biomechanical operation of the facet joint, or restore articulation of the target joint. In another embodiment of the invention, the devices are designed to treat degenerating or diseased tissue in the target facet joint. In yet another embodiment, the device is adapted to restore or maintain motion or mobility for the target facet joint. Objectives of the embodiments can be achieved by, for example, adapting either of the first or second surfaces to conform to an opposing mating surface, or adapting the surface to contour to an opposing mating surface.

Another embodiment of the invention includes a facet capsule device comprising a body adapted to circumvent a superior facet and an opposing inferior facet of a facet joint, wherein the body comprises a flexible body with a first securable edge and a second securable edge adapted to engage the first securable edge. The facet capsule device can be adapted to provide a first securable edge and a second securable edge having apertures for engaging a tying device. In yet another embodiment, the body of the facet capsule device can further be secured by engaging the first securable edge and the second securable edge. The body of the facet capsule replacement device can be configured in some embodiments to be secured to one of the superior facet and the inferior facet by engaging an upper edge of the body or a lower edge of the body to one of the superior facet and the inferior facet. Additional embodiments of the invention can be configured to retain therapeutic materials in contact with a surface of the superior facet or inferior facet or to provide a delivery device for delivering therapeutic materials.

Further embodiments of the invention include a variety of methods. One such method is a method for treating a facet joint comprising: accessing a target facet surface; selecting a facet joint restoration device; and positioning the selected facet restoration device on the target facet surface. Additionally, the embodiments of the method can include the step of deploying a facet joint immobilization device. Still further embodiments, can employ the step of selecting a facet capsule replacement device and implanting or deploying the facet capsule replacement device. Methods can also include the delivery of therapeutic materials to the facet joint or the facet surfaces, or delivery of materials on a time-released basis.

In yet another embodiment of a method according to the invention, a method for treating a facet joint comprising: accessing a target facet surface of a joint; selecting a capsule replacement device; and positioning the selected capsule replacement device on the target facet surface. Embodiments of the method can also include the step of delivering therapeutic materials to the facet joint, or delivering therapeutic materials on a time-released basis. Further, in some instances, it may be desirable to deploy facet joint immobilization devices when practicing the methods of the invention.

In yet another embodiment of the invention, a kit is provided for treating pathologies of the spinal facet, the kit comprising one or more of a facet restoration device, a facet capsule device, a facet immobilization device, and a delivery device for delivering therapeutic materials.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a lateral elevation view of a normal human spinal column;

FIG. 2 is a superior view of a normal human lumbar vertebra;

FIG. 3A is a lateral elevational view of two vertebral bodies forming a functional spinal unit;

FIG. 3B is a posterior view of two vertebral bodies forming a functional spine unit and illustrating a coronal plane across a facet joint;

FIG. 4A is a posterolateral oblique view of a vertebrae from a human spinal column;

FIG. 4B is a posterior view of a vertebra from a human spinal column;

FIG. 5 is a perspective view of the anatomical planes of the human body;

FIG. 6 is a cross-sectional view of a single facet joint in a spinal column taken along a coronal plane;

FIGS. 7A through 7D are various embodiments of facet joint restoration devices constructed in accordance with various teachings of the present invention;

FIG. 8A is a plan view of a facet capsule replacement device constructed in accordance with the various teachings of the present invention;

FIG. 8B; is a perspective view of the facet capsule replacement device of FIG. 8A, in a deployed condition;

FIGS. 9A-C are cross-sectional views of the facet joint of FIG. 6 depicting resection of a facet joint in preparation for implantation, after implantation of a facet joint replacement of FIG. 7; and after implantation of a facet capsule replacement device of FIG. 8;

FIG. 10 is a perspective view of a functional spine unit implanted with the facet capsule replacement device of FIG. 8; and

FIG. 11 is a flow chart illustrating a method according to the invention.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The invention relates generally to implantable devices, apparatus or mechanisms that are suitable for implantation within a human body to restore, augment, and/or replace soft tissue and/or connective tissue, including bone and cartilage, and systems for treating the anatomic or functional manifestation of a disease, such as spinal pathologies. In some instances, the implantable devices can include devices designed to replace missing, removed, or resected body parts or structure. The implantable devices, apparatus or mechanisms are configured such that the devices can be formed from parts, elements or components which alone or in combination comprise the device. The implantable devices can also be configured such that one or more elements or components are formed integrally to achieve a desired physiological, operational or functional result such that the components complete the device. Functional results can include the surgical restoration and functional power of a joint, controlling, limiting or altering the functional power of a joint, and/or eliminating the functional power of a joint by preventing joint motion. Portions of the device can be configured to replace or augment existing anatomy and/or implanted devices, and/or be used in combination with resection or removal of existing anatomical structure.

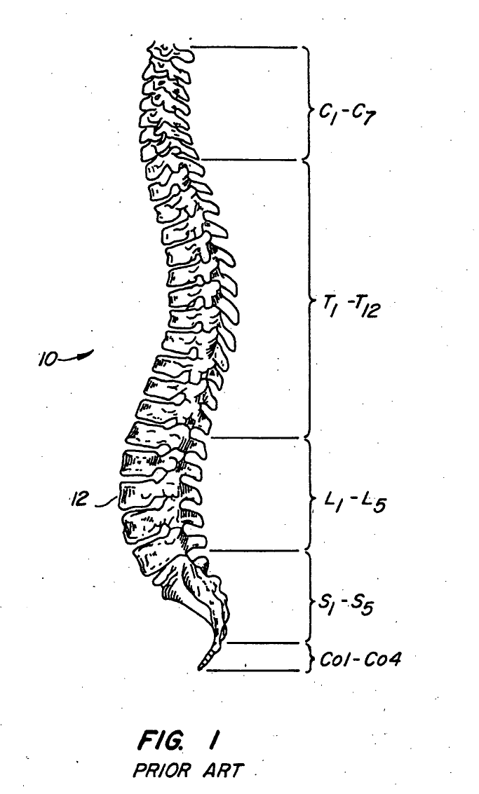

The devices of the invention are designed to interact with the human spinal column 10, as shown in FIG. 1, which is comprised of a series of thirty-three stacked vertebrae 12 divided into five regions. The cervical region includes seven vertebrae, known as C1-C7. The thoracic region includes twelve vertebrae, known as T1-T12. The lumbar region contains five vertebrae, known as L1-L5. The sacral region is comprised of five fused vertebrae, known as S1-S5, while the coccygeal region contains four fused vertebrae, known as Co1-Co4.

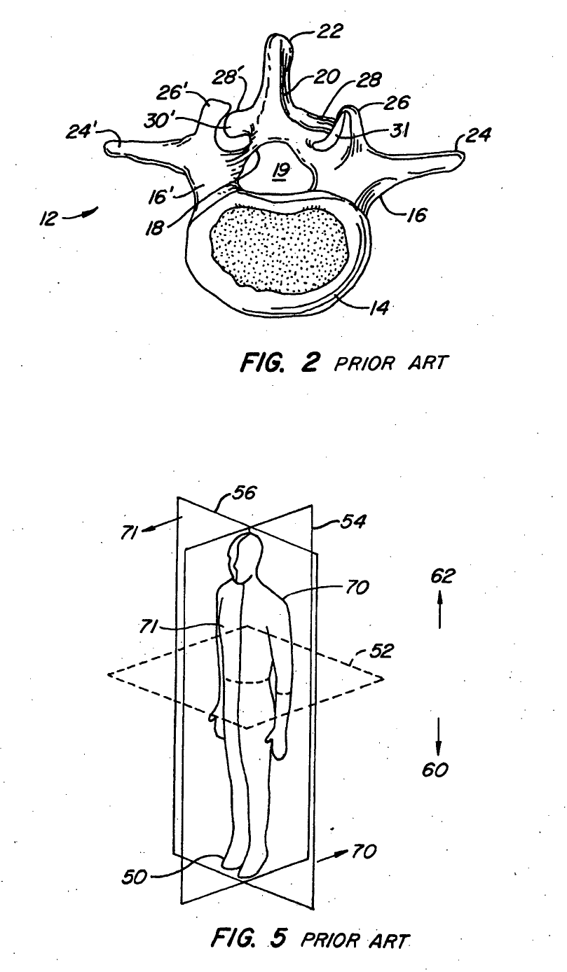

An example of one vertebra is illustrated in FIG. 2 which depicts a superior plan view of a normal human lumbar vertebra 12. Although human lumbar vertebrae vary somewhat according to location, the vertebrae share many common features. Each vertebra 12 includes a vertebral body 14. Two short boney protrusions, the pedicles 16, 16′, extend dorsally from each side of the vertebral body 14 to form a vertebral arch 18 which defines the vertebral foramen 19.

At the posterior end of each pedicle 16, the vertebral arch 18 flares out into broad plates of bone known as the laminae 20. The laminae 20 fuse with each other to form a spinous process 22. The spinous process 22 provides for muscle and ligamentous attachment. A smooth transition from the pedicles 16 to the laminae 20 is interrupted by the formation of a series of processes.

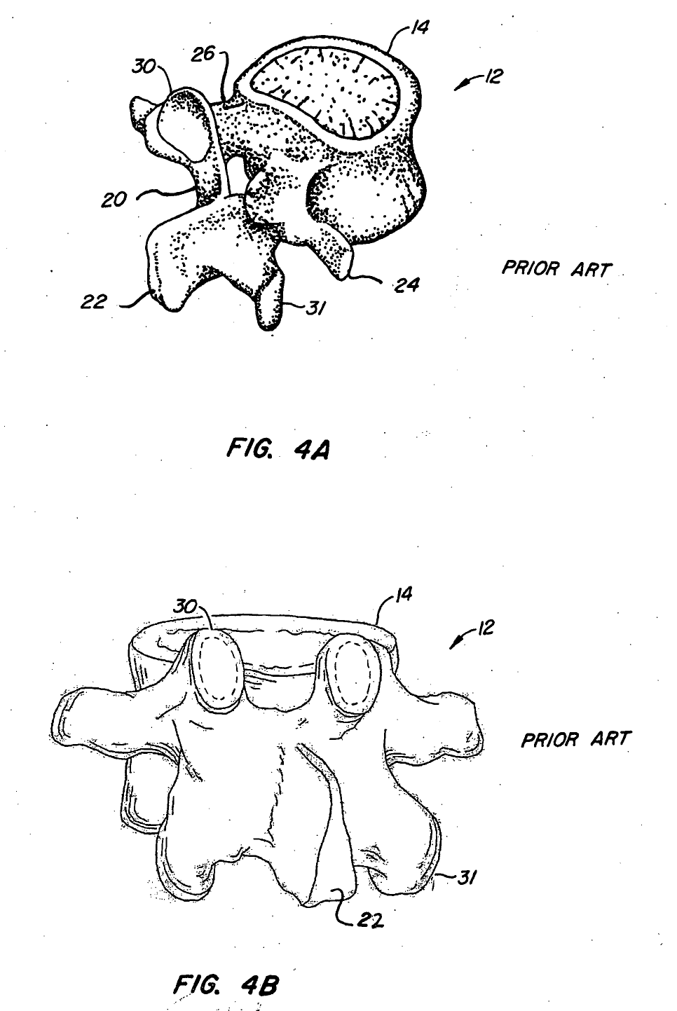

Two transverse processes 24, 24 thrust out laterally, one on each side, from the junction of the pedicle 16 with the lamina 20. The transverse processes 24, 24′ serve as levers for the attachment of muscles td the vertebrae 12. Four articular processes, two superior 26, 26′ and two inferior 28, 28′, also rise from the junctions of the pedicles 16 and the laminae 20. The superior articular processes 26, 26′ are sharp oval plates of bone rising upward on each side of the vertebrae, while the inferior processes 28, 28′ are oval plates of bone that jut downward on each side. See also FIG. 4.

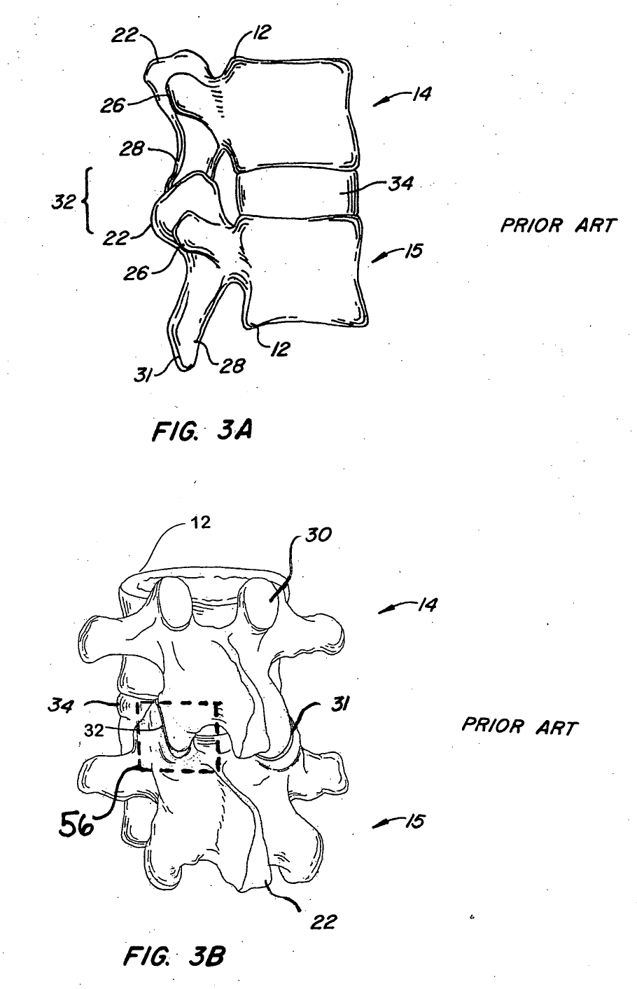

The superior and inferior articular processes 26 and 28 each have a natural bony structure known as a facet. The superior articular facet 30 faces medially upward, while the inferior articular facet 31 (see FIGS. 3 and 4) faces laterally downward. When adjacent vertebrae 12 are aligned, the facets 30 and 31, capped with a smooth articular cartilage and encapsulated by ligaments, interlock to form a facet joint 32. The facet joints are apophyseal joints that have a loose capsule and a synovial lining.

As discussed, the facet joint 32 is composed of a superior facet and an inferior facet. The superior facet is formed by the vertebral level below the joint 32, and the inferior facet is formed in the vertebral level above the joint 32. For example, in the L4-L5 facet joint shown in FIG. 3, the superior facet of the joint 32 is formed by bony structure on the L5 vertebra (i.e., a superior articular surface and supporting bone 26 on the L5 vertebra), and the inferior facet of the joint 32 is formed by bony structure on the L4 vertebra (i.e., an inferior articular surface and supporting bone 28 on the L4 vertebra). The angle formed by a facet joint located between a superior facet and an inferior facet changes with respect to the midline depending upon the location of the vertebral body along the spine 10 (FIG. 1). The facet joints do not, in and of themselves, substantially support axial loads unless the spine is in an extension posture (lordosis). As would be appreciated by those of skill in the art, the orientation of the facet joint for a particular pair of vertebral bodies changes significantly from the thoracic to the lumbar spine to accommodate a joint's ability to resist flexion-extension, lateral bending, and rotation.

An intervertebral disc 34 between each adjacent vertebra 12 (with stacked vertebral bodies shown as 14, 15 in FIGS. 3A-B) permits gliding movement between the vertebrae 12. The structure and alignment of the vertebrae 12 thus permit a range of movement of the vertebrae 12 relative to each other. FIG. 4 illustrates a posterolateral oblique view of a vertebrae 12, further illustrating the curved surface of the superior articular facet 30 and the protruding structure of the inferior facet 31 adapted to mate with the opposing superior articular facet. As discussed above, the position of the inferior facet 31 and superior facet 30 varies on a particular vertebral body to achieve the desired biomechanical behavior of a region of the spine.

Thus, the overall spine comprises a series of functional spinal units that are a motion segment consisting of two adjacent vertebral bodies (e.g., 14, 15 of FIGS. 3A-B), the intervertebral disc (e.g., 34 of FIGS. 3A-B), associated ligaments, and facet joints (e.g., 32 of FIGS. 3A-B). See, Posner, I, et al. A biomechanical analysis of the clinical stability of the lumbar and lumbrosacral spine. Spine 7:374-389 (1982).

As previously described, a natural facet joint, such as facet joint 32 (FIGS. 3A-B), has a superior facet 30 and an inferior facet 31 (shown in FIGS. 4A-B). In anatomical terms, the superior facet of the joint is formed by the vertebral level below the joint, which can thus be called the “caudal” portion of the facet joint because it is anatomically closer to the tail bone or feet of the person. The inferior facet of the facet joint is formed by the vertebral level above the joint, which can be called the “cephalad” portion of the facet joint because it is anatomically closer to the head of the person. Thus, a device that, in use, replaces the caudal portion of a natural facet joint (i.e., the superior facet 30) can be referred to as a “caudal” device. Likewise, a device that, in use, replaces the cephalad portion of a natural facet joint (i.e., the inferior facet 31) can be referred to a “cephalad” device.

As will be appreciated by those skilled in the art, it can be difficult for a surgeon to determine the precise size and/or shape necessary for an implantable device until the surgical site has actually been prepared for receiving the device. In such case, the surgeon typically can quickly deploy a family of devices possessing differing sizes and/or shapes during the surgery. Thus, embodiments of the spinal devices of the present invention include modular designs that are either or both configurable and adaptable. Additionally, the various embodiments disclosed herein may also be formed into a “kit” or system of modular components that can be assembled in situ to create a patient specific solution. As will be appreciated by those of skill in the art, as imaging technology improves, and mechanisms for interpreting the images (e.g., software tools) improve, patient specific designs employing these concepts may be configured or manufactured prior to the surgery. Thus, it is within the scope of the invention to provide for patient specific devices with integrally formed components that are pre-configured.

The devices of the present invention are configurable such that the resulting implantable device is selected and positioned to conform to a specific anatomy or desired surgical outcome. The adaptable aspects of embodiments of the present invention provide the surgeon with customization options during the implantation or revision procedure. It is the adaptability of the present device systems that also provides adjustment of the components during the implantation procedure to ensure optimal conformity to the desired anatomical orientation or surgical outcome. An adaptable modular device of the present invention allows for the adjustment of various component-to-component relationships. One example of a component-to-component relationship is the rotational angular relationship between an anchoring device and the device to be anchored. Other examples of the adaptability of modular device of the present invention as described in greater detail below. Configurability may be thought of as the selection of a particular size of component that together with other component size selections results in a “custom fit” implantable device. Adaptability then can refer to the implantation and adjustment of the individual components within a range of positions in such a way as to fine tune the “custom fit” devices for an individual patient. The net result is that embodiments of the modular, configurable, adaptable spinal device and systems of the present invention allow the surgeon to alter the size, orientation, and relationship between the various components of the device to fit the particular needs of a patient during the actual surgical procedure.

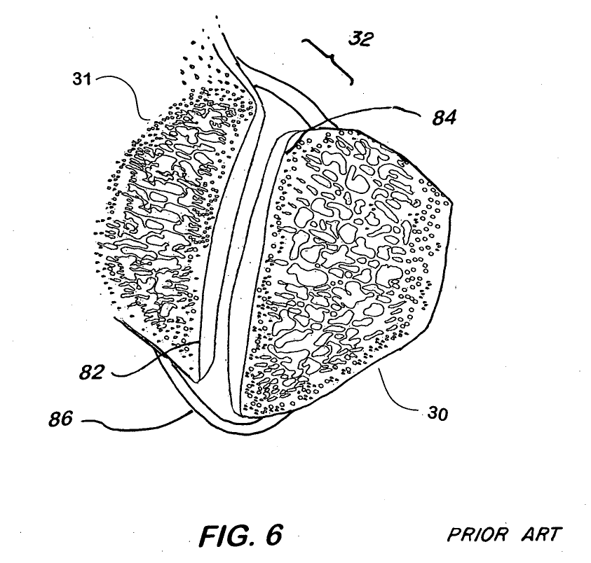

In order to understand the configurability, adaptability and operational aspects of the invention, it is helpful to understand the anatomical references of the body 50 with respect to which the position and operation of the devices, and components thereof, are described. There are three anatomical planes generally used in anatomy to describe the human body and structure within the human body: the axial plane 52, the sagittal plane 54 and the coronal plane 56 (see FIG. 5). Additionally, devices and the operation of devices are better understood with respect to the caudal 60 direction and/or the cephalad direction 62. Devices positioned within the body can be positioned dorsally 70 (or posteriorly) such that the placement or operation of the device is toward the back or rear of the body. Alternatively, devices can be positioned ventrally 72 (or anteriorly) such that the placement or operation of the device is toward the front of the body. Various embodiments of the spinal devices and systems of the present invention may be configurable and variable with respect to a single anatomical plane or with respect to two or more anatomical planes. For example, a component may be described as lying within and having adaptability in relation to a single plane. For example, an anchoring device may be positioned in a desired location relative to an axial plane and may be moveable between a number of adaptable positions or within a range of positions. Similarly, the various components can incorporate differing sizes and/or shapes in order to accommodate differing patient sizes and/or anticipated loads.

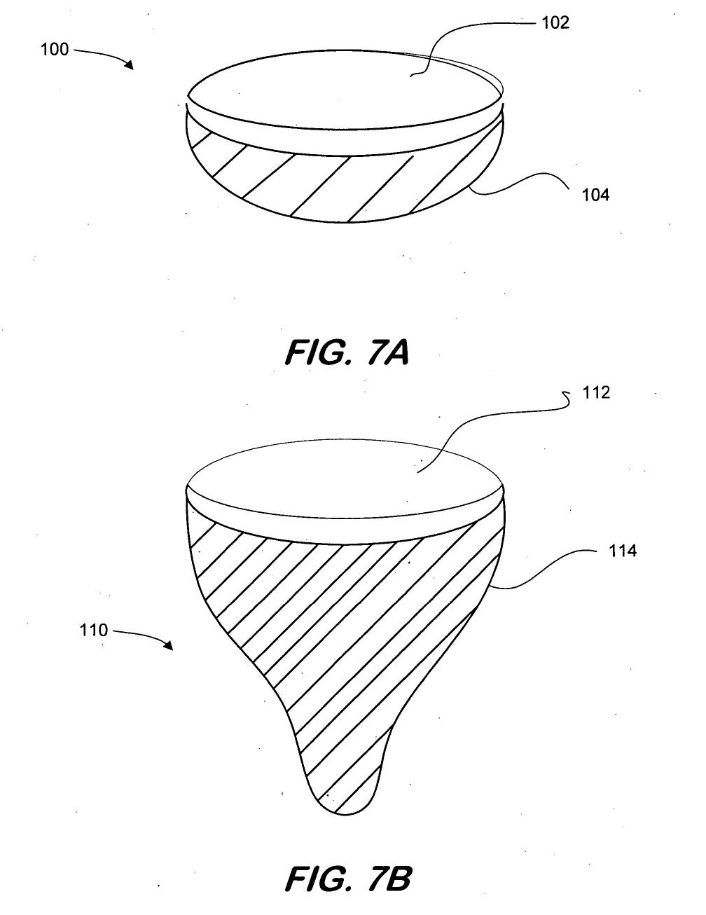

FIG. 6 depicts a facet joint 32 in cross-section along, for example, a coronal plan (56 of FIGS. 3B and 5). As will be appreciated, the orientation of a facet joint in any plane of the body changes depending upon the location of a particular joint within the spinal column, this example is provided for illustration purposes only.

The facet joint 32, is formed from a superior articular facet 30 and an inferior articular facet 31. The inferior articular facet 31 has a cephalad facet surface 82 and the superior articular facet 30 has a caudal facet surface 84. Normally, each of the surfaces has an articulating cartilage layer positioned adjacent the facet surfaces 82, 84 to improve the movement of the facet joint 32 in operation. In addition to the caudal facet surface 84 and the cephalad facet surface 82 that comprise the opposing joint surfaces, each of the superior articular facet 30 and the inferior articular facet 31 have facet surfaces on the sides of the facets. A facet capsule 86 is also provided that surrounds the facet joint 32 and to communicate with the facet surfaces on the sides of the superior articular facet 30 and the inferior articular facet 31. Where the anatomic or functional manifestations of a disease has resulted in a spinal pathology, facet joint degradation can occur, which includes wear of the articulating surface of the facet joint. Normally, the peripheral, cortical rim of the joint is not affected, or is minimally affected. With hypertrophic facets, the mass of cortical bone and action of the osteophytes can make the facet larger than normal as the facet degenerates. When a facet begins to wear, the biomechanics of the functional spine unit are altered, which causes further damage to the facet joint and pain.

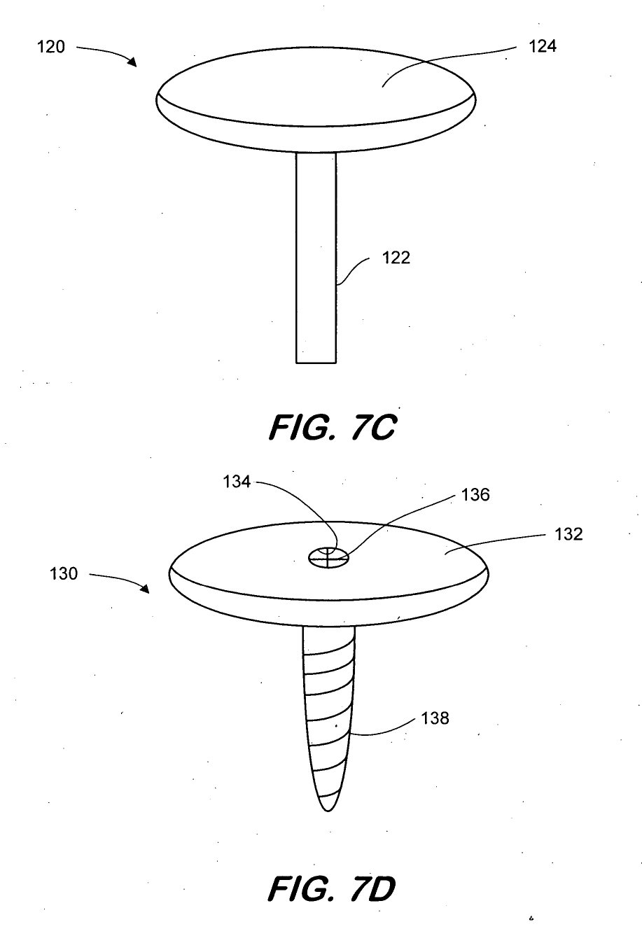

Turning now to FIG. 7, a variety of facet surface repair and restoration devices or plugs are depicted. Embodiments of the facet surface repair devices could include almost any shape and/or size plug that can fit within the space created by resecting the facet joint, or otherwise preparing the joint surface, if necessary, for restoration. Various shapes can include, for example, a facet surface repair or restoration device 100 having a first surface 102 adapted to recreate or restore the damaged facet surface and a second surface 104 adapted to conform to a resected or damaged facet surface as shown in FIG. 7A. The second surface 104 can be altered to promote adhesion to the facet surface, e.g., by providing teeth, anchors, ridges, nubs, serrations, a roughened or nubby finish (e.g., by sandblasting to achieve a granulated surface), or coatings that promote bony in-growth. Alternatively, a facet surface repair device 110 can be configured to achieve a shape similar to a chalice as illustrated in FIG. 7B. Similar to the device in FIG. 7A, a first surface 112 is provided to mate with the opposing surface of the facet joint and a second surface 114 is provided to conform to the target surface to be repaired. The first surface 102, 112 is depicted as a flat surface but, as would be appreciated by those skilled in the art, could take a variety of surface configurations including, but not limited to, convex, concave, variable, conforming, etc.

In yet another embodiment, the facet surface restoration device 120 is configured similar to a tack, golf tee, or a nail with a stem 122, optionally having a sharp point, and a head 124 as depicted in FIG. 7c. The head 124 can be configured to present a mating surface for a facet joint. The stem 122 can be configured to promote adhesion of the device to the bone. In yet another embodiment of the facet surface restoration device 130 shown in FIG. 7D a plate 132 is provided having an aperture 134 for receiving a screw 136. The screw has a threaded anchoring shaft 138 for engaging the facet joint as described above. The upper surface of the plate 132 can be configured to provide an optimal mating surface for a mating facet joint. Of course, as will be appreciated by those skilled in the art, whichever form the device takes, embodiments can be configured to fit within the surrounding cortical bone rim, which can further secure the device in place. If desired, additional securing means can be used to secure the device within the facet joint, including, but not limited to, screws, press-fit stems, surface alterations, surface coatings, etc.

The facet restoration device could also be configured to incorporate a modular stem/surface combination, allowing for replacement of the first surface (e.g., 102, 112) to restore the facet joint, if necessary. Such a configuration would be useful where, for example, the facet joint experienced excessive surface wear over time (allowing for replacement of the worn surface with a new surface or one incorporating a different design), or to allow removal and/or revision of the device to achieve fusion. If desired, the modular stem may be retained with the facet joint and used to subsequently anchor a fusion device. Desirably, the implant will be retained within the facet joint simply by the natural compressive forces of the joint, combined with retention by the surrounding cortical bone rim. Of course, additional integration into the facet joint, either through the use of mechanical fasteners, or biological in growth, could be accomplished with varying results. Each of the devices can be configured as a single piece or more than one piece that is assembled to achieve the final arrangement.

A variety of materials are suitable for making the device illustrated in FIG. 7, including but not limited to, Nickel-Titanium alloys and other metals and shape memory metals, ceramics and polymers. The bone restoration devices can also be configured to comprise a cancellous/cortical bone base topped by an associated layer of articulating cartilage or other articulating material. Various sources of suitable bone restoration devices can be obtained through methods well known in the orthopedic arts; including bone harvesting procedures such as osteochondral grafting, using either the patient's own tissues (autograft), or a matched or processed graft from another source (allograft or xenograft). Other sources of such grafts could potentially be autologous chondrocyte implantation (mature cartilage cell injection) or mesenchymal stem cell implantation. In various alternate embodiments, the restoration device could comprise artificial materials, such as, for example, a moldable, extrudable and/or settable material, which could be contoured to surrounding cortical bone in situ (or just immediately prior to implantation), or could be prepared prior to the surgical procedure (allowing for precise contouring to the surrounding cortical bone and/or allowing for congruent articulation such that a first joint surface is positioned such that it is superimposed, or substantially superimposed, with respect to the opposing facet joint surface). One or more facet joint surfaces of a target facet joint can be the natural surface (i.e., the surface present in the joint), or can be restored with naturally derived or artificial materials.

Alternatively, the replacement material used could comprise a ceramic, polymer or other biocompatible material with articulating characteristics similar to facet cartilage, or which articulates well with the opposite facet surface material if both sides of the facet are replaced. If desired, the surface of the restoration device in contact with the cancellous bone of the facet can comprise a material allowing biological in growth, which could be a different material than the articulating material on the outer face of the plug. Suitable polymers would be known in the art and include, but are not limited to, a polyketone known as polyetheretherketone (PEEK™). Where a polymer is used, the device can be formed by extrusion, injection, compression molding and/or machining techniques. This material has appropriate physical and mechanical properties and is suitable for carrying and spreading the physical load between the facet joint. It should be noted that the material selected may also be filled. For example, other grades of PEEK are also available and may be suitable, such as 30% glass-filled or 30% carbon filled, provided such materials are suitable for use in implantable bodies, including, but not limited to, those cleared for use in implantable devices by the FDA, or other regulatory body. Glass filled PEEK reduces the expansion rate and increases the flexural modulus of PEEK relative to that which is unfilled. The resulting product is known to be ideal for improved strength, stiffness, or stability. Carbon filled PEEK is known to enhance the compressive strength and stiffness of PEEK and lower its expansion rate. Carbon filled PEEK offers wear resistance and load carrying capability. As will be appreciated, other suitable similarly biocompatible thermoplastic or thermoplastic polycondensate materials that resist fatigue, have good memory, are flexible, and/or deflectable have very low moisture absorption, and good wear and/or abrasion resistance, can be used without departing from the scope of the invention. Additional suitable materials include polyetherketoneketone (PEKK), polyetherketone (PEK), polyetherketoneetherketoneketone (PEKEKK), and polyetheretherketoneketone (PEEKK), and generally a polyaryletheretherketone. Further other polyketones can be used as well as other thermoplastics.

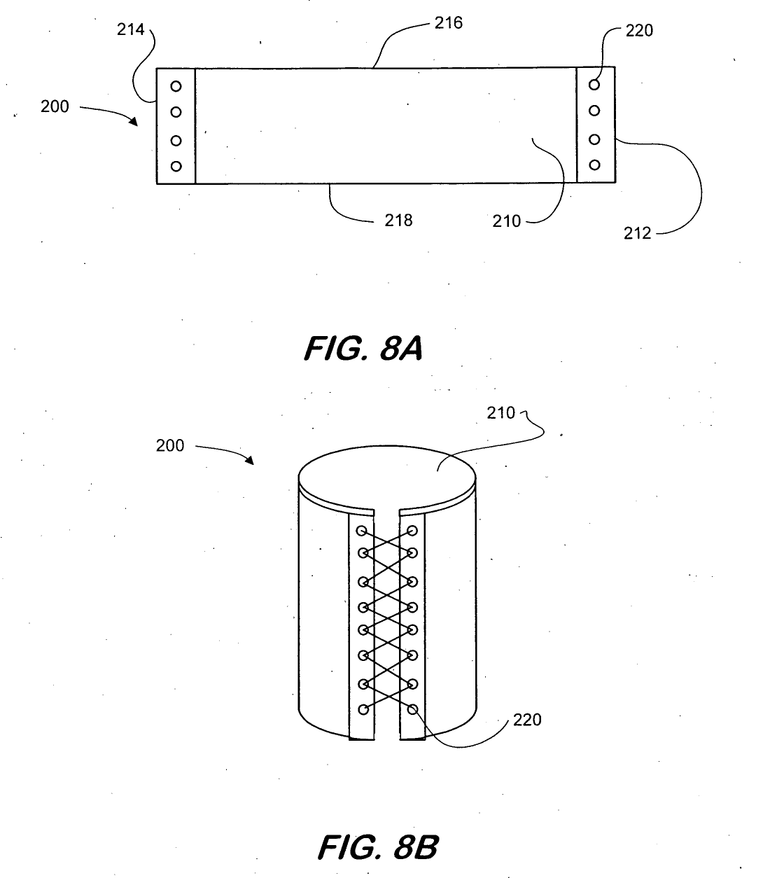

Turning now to FIGS. 8A-B, an alternate facet treatment device is depicted. The facet capsule replacement device 200 is adapted to address an underlying facet pathology involving damage to the facet capsule, or where the facet capsule has been compromised in some manner, such as to allow access to the facet joint for some reason. The disclosed embodiment can be used to repair, augment and/or replace some or all of the facet capsule.

The capsule replacement device 200 comprises a flexible and/or elastomeric body 210. Suitable materials for the flexible, elastomeric body include, for example, a biocompatible polymeric material. Securing holes 220 are provided at a first end 212 and a second end 214 of the body 210. In use, the body 210 is positioned around the facet joint 32, and the ends 212, 214 are secured to each other as depicted in FIG. 8B, thereby wrapping around and encapsulating the superior and inferior facets, which may be intact, repaired, resurfaced and/or replaced on one or both sides. The device 200 may be capable of retaining one or more therapeutic materials in contact with any of the surfaces of the superior or inferior facet, such as fluids, to promote articulation, pain relief, cell regeneration, or other use, as would be appreciated by those skilled in the art. Where the device 200 retains materials for contact with the surfaces, the objective can be achieved by including a biocompatible adhesive to adhere the edges of the device 200 to the inferior facet and/or superior facet. Alternatively, the device 200 can be comprised of a plurality of layers that enable therapeutic materials to be delivered on a time-released basis to the target surface. Thus, for example, an external layer could act as a barrier while an interior layer could be embedded with therapeutic materials for delivery to a facet surface. A plurality of inner layers could also be provided which are individually adapted to deliver a therapeutic material. An additional delivery device can be employed in combination with the devices disclosed herein to deliver therapeutic material. Such delivery device can include, for example, a layer of material that is embedded with therapeutic material for delivery to a target site. The layer is positioned on the target site and the devices are deployed.

The upper edge 216 and the lower edge 218 of the device 200 can be secured to the outer surface of the respective facet joint with which the edges are in contact. For example, the upper 216 and lower 218 edges of the device 200 can be configured to incorporate metal hooks which could be used to secure the device 200 to the outer surface of the facet joint (directly to the bone, to the pre-existing facet capsule material, or both). Alternatively, the upper and lower edges could be sutured to the surrounding anatomy in a known manner. Of course, the device 200 could be used in concert with repair and/or replacement of the facet joints and facet joint surfaces using the devices illustrated in FIG. 7, or can be used independent of such repair.

In various alternative embodiments, the inner surface(s) of the device 200 could incorporate materials or drugs which promote adhesion between the facet capsule and the surrounding flexible body 210. These embodiments would allow the body to adhere to the remaining facet capsule, thereby securing the device 200 to the facet, allowing it to augment the remaining strength of the facet capsule, and potentially sealing the facet capsule against leakage of fluids. Similarly, the ends 212, 214 of the device 200 could comprise similar materials, or could include osteoconductive and/or osteoinductive materials to promote fusion of the ends to the surround cortical and/or cancellous bone structures. Additionally a reinforcing layer can be provided. A reinforcing layer can be formed from appropriate mechanical or materials including, for example, rods, spines, stents, wires, or other supports could be incorporated into the body to further reinforce the device while retaining flexibility during and/or subsequent to deployment.

By reinforcing and/or replacing the facet capsule, the present invention will desirably assist the facets in F controlling and/or limiting movement between the vertebrae, thereby protecting the intervertebral discs from shear forces, excessive flexion and/or axial rotation. In addition, it may be desirable to alter the biomechanics of the facet joint to accomplish some desired result (allowing more, less or modified motion across one or more of the facet joints). For example, where annular bulging has or is occurring, it may be desirous to reduce facet motion at that level, either by itself or in combination with treatment of the affected intervertebral disc of that vertebral level. In addition, it may be desirous to only temporarily alter facet motion to allow for natural healing, then to resume normal motion after healing has occurred.

The disclosed facet capsule device could also be utilized to reinforce the facets (and thus the entire functional spinal unit) against unwanted spondylolitic slip. Alternatively, the facet capsule device could be positioned around the natural location of a natural facet joint, or could be implanted at a location adjacent to or remote from the actual location of the natural facet capsule, such as between adjacent mamillary processes on adjacent vertebrae (or around adjacent spinous processes). In other embodiments, the facet capsule device could be used to retain and hold a free-floating or semi-anchored spacer (not shown) which might separate the two facet surfaces from each other.

Depending upon the design and material choice for the facet capsule replacement device 200 (as previously noted), the device may be capable of sealing and/or retaining fluids within the facet joint. Such a device would be especially well suited to reseal a damaged facet capsule (thereby allowing replacement and/or retention of remaining synovial fluid within the facet joint) or could be suited to retain materials that promote cell regeneration or relieve pain in the facet joint and/or surrounding anatomy.

In a similar manner, the facet capsule replacement device could be altered to be useful in repairing, creating, replacing and/or augmenting any other joints of the body, including, but not limited to, joints such as those found in the temporomandibular, sternocostal, hip, knee, shoulder, clavicle, glenoid, elbow, wrist, metacarpal, ankle and/or metatarsal areas.

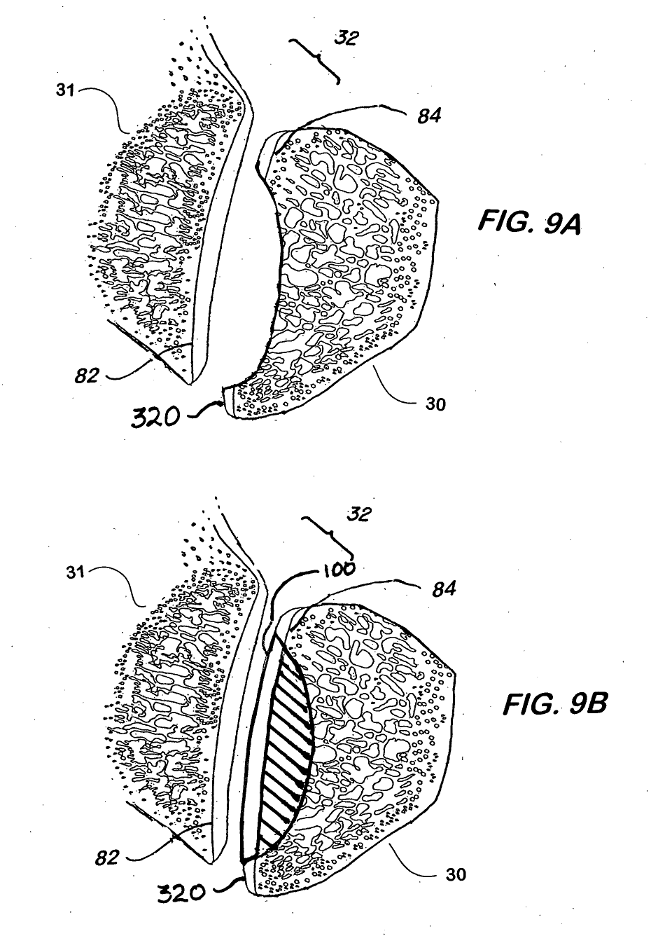

Turning now to the methods of practicing the invention, as illustrated in FIG. 9 the facet capsule(s) surrounding the facet joint(s) 32 can first be resected or prepared in a known fashion, the result of which is shown in FIG. 9A. Typically, resection will be minimal, to allow for the resection to be subsequently closed at the conclusion of the surgery. However, more extensive resection may be appropriate and/or desirable under some conditions. Resection results in the interior facet joint surfaces being accessed. Once the facet capsule 86 (shown in FIG. 6) has been resected the facet joint surfaces 82, 84 are accessed. Depending on the state of the disease, one or more of the facet joint surfaces can be resected. FIG. 9A illustrates the resection of the caudal facet joint surface to form a target resected area 310 for repair. As a result of the resection, the facet surface is prepared for receiving an artificial or natural facet replacement device (e.g., the devices shown in FIG. 7) by removing the overlying cartilage, as well as a section of the underlying cancellous bone. A circumferential rim 320 of cortical bone surrounding the resected facet joint surface can be left intact. Alternatively, or in addition to the above-described procedure, a similar procedure can be utilized to prepare and/or treat the cephalad facet joint surface.

To remove the facet material, a surgical rongeur or wedge/cutter (not shown) may be inserted between the articulating surfaces of the facet joint, and positioned using direct visualization and/or non-invasive visualization technology (including, but not limited to, x-ray, fluoroscopy, real time MRI, etc.). The wedge/cutter can be used to resect a significant portion (or all) of the articulating surface of one half of the facet joint, along with an associated bone plug, while simultaneously preparing the half of the facet joint for the replacement facet plug.

After the caudal facet surface is prepared, an appropriately sized facet bone restoration device, such as bone restoration devices 100, 110, 120, 130 illustrated in FIGS. 7A-D, is placed into the prepared space in the caudal facet joint as shown in FIG. 9B. The bone restoration device can be positioned such that it is inset or inlayed in the target facet joint. As illustrated, the device fits within an intact bone cortical rim 320. However, as will be appreciated by those skilled in the art, the same results can be achieved without the requirement of an intact bone cortical rim 320.

In the case where replacement/treatment of both upper and lower facet surfaces of a individual facet joint halves is desired, both facet surfaces can be resected and prepared for implantation, with the replacement facet plugs introduced into the prepared depressions. If desired, the plugs can be secured into a desired position (using cement, pins, etc) or held in position by compressive force with a shield (such as a non-reactive sheet of Tyvek®, etc.) positioned between the upper and lower facet surfaces, so as to allow the facets to biologically secure to the surrounding tissues.

As part of the facet replacement procedure, the treated facet joint(s) may be immobilized for a pre-determined period of time to allow the facet plugs to bond with the surrounding tissues, or the facet can be mobilized immediately to prevent scarification resulting from the mechanical abrasion which could occur with joint motion, and/or unwanted arthrodesis across the facet joint. If desired, a sheet of non-bio-active material can be placed between the upper and lower surfaces of the facet for a period of time to prevent arthrodesis across the facet joint, with the eventual removal (or natural or chemically induced degradation) of the material after desired healing has occurred.

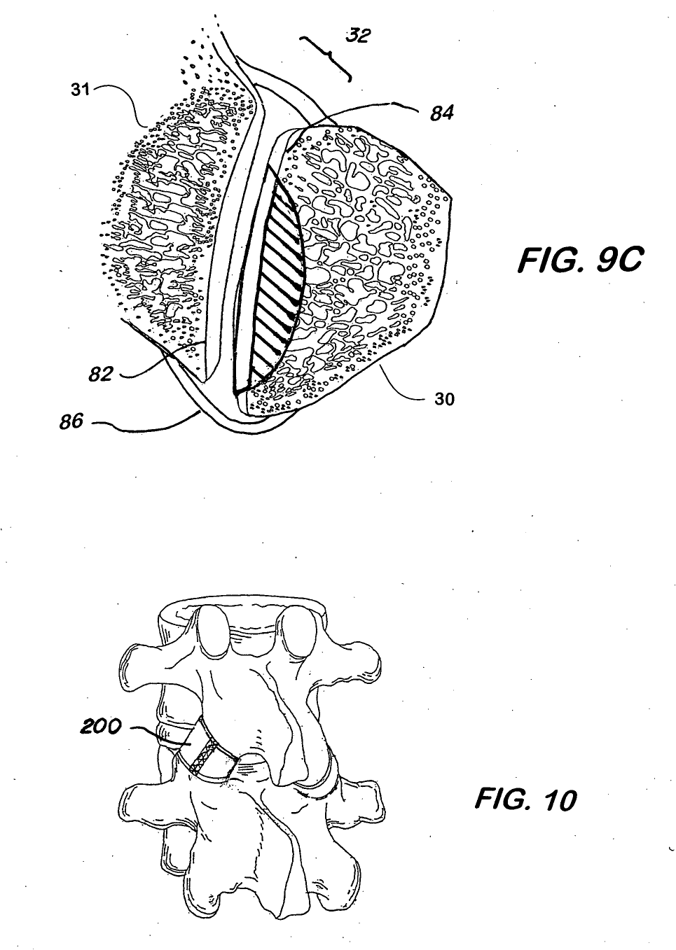

Additionally, a device, such as the capsule replacement device 200 can be incorporated in addition or in place of Tyvek as shown in FIG. 9c. FIG. 10 illustrates a posterior view of a pair of vertebral bodies having a capsule replacement device 200 associated therewith.

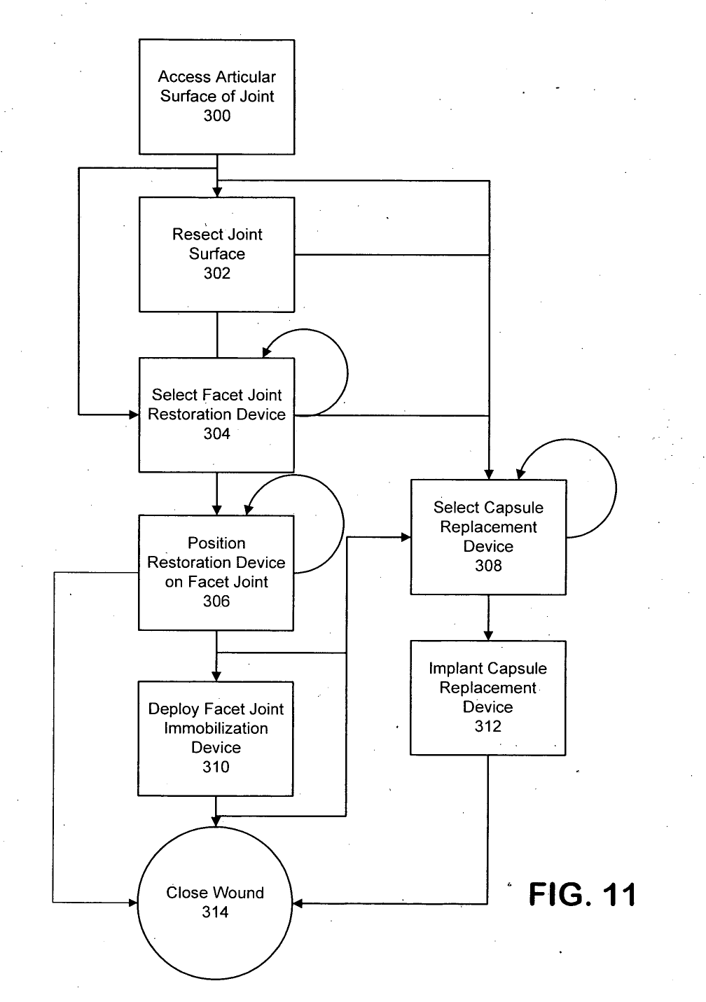

FIG. 11 illustrates a flow chart of the method described above with respect to FIGS. 9-10 using the devices of FIGS. 7-8. Initially, the target articular surface of a joint is accessed 300. If necessary, the joint surface is resected 302. Thereafter, the physician can select a facet joint restoration device 304 or select a capsule replacement device 308, as desired or necessary. Each of these steps can be repeated, if required or desirable. Once the facet joint restoration device is selected 304, the device is positioned within the facet joint 306. Repositioning of the device can also be performed, if desired. Thereafter, the wound can be closed 314 or a facet joint immobilization device can be selected and deployed 310. Following the step of positioning the restoration device 306 and/or deploying the immobilization device 310, the surgeon can also optionally proceed with the step of selecting a capsule replacement device 308, as described above. Once a capsule replacement device is selected, it is then implanted 312 and the wound is closed 314. As will be appreciated by those skilled in the art, implanting the capsule replacement device 312 can also occur without accessing the facet joint surface (or even without resecting the facet joint capsule itself) before the deployment of the joint immobilization device 310 without departing from the scope of the invention.

In various embodiments, there is provided a modular component kit or system and an associated surgical method of selecting from the component kit configurable elements that, separately and in combination, provide an adaptable devices corresponding to the anatomical needs. The kit provides a variety of various sized device components. The method includes selecting components from the kit having the desired size, angular orientation and anatomical orientation that correspond to the needs of the patient. In additional embodiments, there is provided a method of adapting the devices to an individual's anatomy wherein the adaptability is achieved by selecting from a subset of different sizes and configurations of components.

Although various specific embodiments have been disclosed herein, it should be understood that many alternatives, modifications and variations will be apparent to those skilled in the art. Accordingly, other alternatives, modifications and/or variations are contemplated that fall within the scope of the following claims.

What is claimed is:

1. A facet joint restoration device for use in a restoring a target facet joint surface comprising:
(a) a first surface configured to articulate with respect to an opposing surface comprising one of a facet joint surface or a facet joint restoration device surface; and
(b) a second surface configured to engage a surface of the target facet joint.

2. The facet joint restoration device according to claim 1 wherein the second surface is configured to promote bony in-growth.

3. The facet joint restoration device according to claim 1 wherein the second surface is adapted to secure the restoration device to the surface of the target facet joint.

4. The facet joint restoration device according to claim 1 wherein the second surface is adapted to provide an anchoring mechanism.

5. The facet joint restoration device of claim 4 wherein the anchoring mechanism includes anchoring mechanisms selected from the group consisting of teeth, ridges, nubs, serrations, granulations, a stem, and a spike.

6. The facet joint restoration device according to claim 1 wherein the device is configured from naturally occurring materials adapted to form the device, ceramic, metal, or polymer, or combinations thereof.

7. The facet joint restoration device according to claim 1 wherein the device restores the biomechanical operation of the facet joint.

8. The facet joint restoration device according to claim 1 wherein the device treats degenerating or diseased tissue in the target facet joint.

9. The facet joint restoration device according to claim 1 wherein the device is adapted to restore or maintain motion or mobility for the target facet joint.

10. The facet joint restoration device according to claim 1 wherein one of the first or second surface is adapted to conform to a mating surface.

11. The facet restoration device according to claim 1 wherein one of the first or second surface is adapted to contour to a mating surface.

12. The facet restoration device according to claim 1 wherein the device restores articulation of the target facet joint.

13. A facet capsule device comprising a body adapted to circumvent a superior facet and an opposing inferior facet of a facet joint, wherein the body comprises a flexible body with a first securable edge and a second securable edge adapted to engage the first securable edge.

14. The facet capsule replacement device of claim 13 wherein the first securable edge and the second securable edge have apertures for engaging a tying device.

15. The facet capsule replacement device of claim 13 wherein the body is secured to the superior facet and inferior facet by engaging the first securable edge and the second securable edge.

16. The facet capsule replacement device of claim 13 wherein the body is secured to one of the superior facet and the inferior facet by engaging an upper edge of the body or a lower edge of the body to one of the superior facet and the inferior facet.

17. The facet capsule replacement device of claim 13 wherein the device is configured to retain therapeutic materials in contact with a surface of the superior facet or inferior facet.

18. The facet capsule replacement device of claim 17 wherein the therapeutic material promotes articulation, adhesion, pain relief, or cell regeneration.

19. The facet capsule replacement device of claim 13 further comprising a reinforcing layer.

20. A method for treating a facet joint comprising:
(a) accessing a target articular surface;
(b) selecting a facet joint restoration device; and
(c) positioning the selected facet restoration device on the target articular surface.

21. The method of claim 20 further comprising the step of deploying a facet joint immobilization device.

22. The method of claim 20 further comprising the step of selecting a facet capsule replacement device and implanting the facet capsule replacement device.

23. The method of claim 22 further comprising the step of delivering therapeutic materials to the facet joint.

24. The method of claim 21 wherein the therapeutic materials are delivered on a time-released basis.

25. The method of claim 20 further comprising the step of deploying a facet joint immobilization device.

26. A method for treating a facet joint comprising:
(a) accessing a target articular surface of a joint;
(b) selecting a capsule replacement device; and
(c) positioning the selected capsule replacement device on the target articular surface.

27. The method of claim 26 further comprising the step of delivering therapeutic materials to the facet joint.

28. The method of claim 27 wherein the therapeutic materials are delivered on a time-released basis.

29. The method of claim 26 further comprising the step of deploying a facet joint immobilization device.

30. A kit for treating pathologies of the spinal facet, the kit comprising one or more devices for treatment of the spinal facet pathology, the devices selected from the group consisting of: a facet restoration device, a facet capsule device, a facet immobilization device, and a delivery device for delivering therapeutic materials.
